(19) United States
(12) Patent Application Publication
(10) Pub. No.: US 2009/0291144 A1
(43) Pub. Date: Nov. 26, 2009

(54) Therapeutic and Prophylactic Compositions Including Catalytic Biomimetic Solids and Methods to Prepare and Use Them

(75) Inventor: Miroslav Colic, Goleta, CA (US)

Correspondence Address: Lance J. Lieberman, Esq.;Cohen, Pontani, Lieberman & Pavane, Suite 1210, 551 Fifth Avenue, New York, NY, 10176, US

(73) Assignee: Henceforth Hibernia, Inc.

(21) Appl. No.: 12/534,555

(22) Filed: Aug. 3, 2009

Related U.S. Application Data
(63) Continuation of application No. 10/790,544, filed on Mar. 1, 2004.
Application No. 10/790,544 is a division of application No. 09/640,218, filed on Aug. 16, 2000, now abandoned.
(60) Provisional application No. 60/149,131, filed on Aug. 16, 1999.

(51) Int. Cl. A61K 9/14; A61K 33/06; A61P 35/00; A61K 33/30; A61K 33/38; A61K 35/00; A61K 39/00; A61K 38/20; A61K 38/18; A61K 38/21

(52) U.S. Cl. 424/489; 424/684; 424/643; 424/618; 424/93.1; 424/277.1; 424/85.2; 514/12; 424/85.5

(57) ABSTRACT

The invention discloses therapeutic and prophylactic compositions based on synthetic solid catalysts such as zeolites, clays, silicates, silicas and double hydroxides. These solids can be used to treat numerous disease conditions such as diabetes, arthritis and other autoimmune diseases, cancer, skin diseases, microbial infections etc. The invention also describes methods to produce such products and use them independently or in combination with other pharmaceutically and biologically active ingredients. Such catalysts are designed so to imitate biological catalytic systems (enzymes, antigen presenting cells, delayed active component release, cell organeles, etc.) and are, therefore, biomimetic.

### FIELD OF THE INVENTION

The invention describes therapeutic and prophylactic compositions based on catalytic biomimetic solid particles such as zeolites or silicas and methods to prepare and use such solids.

### BACKGROUND OF THE INVENTION

Insoluble colloidal particles and powders, such as talc, are routinely used in cosmetics. It was only recently that the bioeffects of internally applied insoluble materials have been described. Inhalation of fibrogenic particles such as asbestos or quartz and result in lung fibrosis, and sometimes cancer. [1] On the other hand, intraperitoneal treatment of animals prone to developing diabetes, such as nonobese diabetic mice (NOD mice), with silica powder, resulted in preventing the appearance of diabetes. [2] Silica powders have also been used in wound healing where it was shown that silica can either enhance or reduce the rate of proliferation of dermal fibroblasts. [3] Zeolite powders have also been used as a vaccine adjuvant. [4] Zeolite powders with zinc or silver inside the pores are efficient antimicrobial agents. [5] Orally applied natural zeolite was also used in treatment of enteritis. [6]

Despite very potent and diverse catalytic activities of such solids, their therapeutic use has been limited due to poor transport into the body and the risk of side effects. Therefore, it is the purpose of this invention to describe solid carrier and catalytic particles designed at the molecular level (nanoengineering) so that transport to target organs/tissues and target activities are maximized, with acceptable or no significant side effects.

Analysis of the cooperative behavior of subunits within a controlled spatial assembly such as membranes or lisosomes is a field of explosive growth. Bioorganic chemistry, an area that deals with nanocomposite biological systems consisting of inorganic and organic constituents, is profiting from new scientific developments in nanotechnology. Nanotechnology is an area of engineering and science that deals with material preparation and modification on molecular or nanoscopic levels. Modifying atomic and nanoscopic supramolecular structures of materials results in new macroscopic properties. Biomimetic chemistry profits knowledge about the functional relationships of biological supramolecular structures. By imitating such natural systems, scientists can design new functional materials with the desired properties.

In this patent we describe a biomimetic approach to synthesize and use catalytic solids with strong experimental therapeutic potential. Supramolecular structures consisting of silicate based solids such as zeolites, organic or metalloorganic entities with catalytic properties and other necessary molecular units, modify bioavailability and/or specific activities of synthesized solids.

A feature of functional proteins and enzymes is their ability to create a reaction space inside the molecule and a specific surface that can be recognized by other functional molecules. The reactive groups of the enzyme and the substrate molecules are organized at the so called active site Silicate based inorganic materials which in their structure resemble such enzymes and functional proteins can be used as the “backbone” of the biomimetic catalytic materials. Zeolites, clays, double hydroxides, silicates and porous silicas are typical examples of such materials.

Porous materials such as zeolites often have some catalytic activity of their own. However, to enhance the therapeutic efficiency of such solids one can nanoengineer the catalytic entities inside the pores to produce the desired effects. This is performed in prior art to produce catalysts for waste water treatment or chemical catalysis. For these applications, larger micron size particles are suitable. For biomedical application, smaller submicron and nanosized pore containing particles are needed for efficient transport inside tissues and organs and for bioavailability. Such particles will be described in this invention.

Catalytic entities are usually encapsulated metal complexes such as Schiff-base complexes, metal porphyrins, phthalocyanines or corrinoids. In this chemistry, the solid particle with its pores/cages is a molecular scale micro or nanoreactor. Entrapped metal complexes within the cages act as catalytic units similar to the active site of enzymes. Other pores in such solid nanoreactors, such as zeolites are of well-defined size and shape so that only molecules of certain size and shape can penetrate. The ligands bound to the metal inside the cage/pore of the catalytic particle can also be engineered to perform specific catalytic reactions. The ligands modify or fine-tune the electronic, stereochemical and structural environment of a metal ion. The encapsulated metal complexes have catalytic properties that are different from those of pure cation exchanged zeolite. Such encapsulated metal complexes also have different catalytic properties than metal complexes dissolved in water or organic solvents. Porous solid nanorectors are actually used to modify catalytic properties of encaged metal complexes, or to release them with time delay.

The surface of such catalytic solids can be modified for enhanced bioavailability without destroying the catalytic activity of the encapsulated metal complex. Inactivation of such metal complexes by dimerization or interaction with large macromolecules is also prevented. Particle size, shape, wettability (hydrophilic or hydrophobic), charge, and stereochemistry as well as the presence of the adsorbed functional molecules can be engineered. Such modifications for therapeutic purposes will be described in this invention. Ideally, functional therapeutic particles should be transported to tissues and organs where they are desired for treatment and excluded from tissues or organs where they may be harmful.

### SUMMARY OF THE INVENTION

As mentioned in the Introduction, particles and insoluble solids have been used in external uses such as skin care. Local therapeutic effects inside the stomach and intestines, such as the treatment of enteritis, were also achieved. Utilizing insoluble particles for therapeutic purposes inside the body (internally other than the GI tract) has not been possible, due to the poor adsorption of such particles.

The purpose of this invention is to describe therapeutic and prophylactic compositions which contain insoluble particles (solids) which can be adsorbed by mucous membranes and by body fluids. Thus, they can be used for internal as well as external treatment of disease.

These particles can be nanoengineered to achieve maximum therapeutic efficiency with minimal side effects. A biomimetic (biomimetic=imitating nature's own solutions) approach is used to synthesize these particles with well-defined pores. Inside the pores, active metal complexes, drugs, macromolecules or whole cells are encapsulated to achieve the desired therapeutic activity. The particle surface is also modified to achieve bioavailability to desired tissues and organs. In particular, particle charge, wettability and the presence of adsorbed active molecules, that modify bioavailability, are engineered. In our approach, submicron and nanoparticles are used to achieve bioavailability for internal (i.e. internal organs other than the GI tract) use. Particles are prepared with high energy ball milling, aqueous hydrothermal synthesis or sol-gel synthesis. Catalytic entities are either encapsulated during synthesis or incorporated later.

These particles are used in three fundamentally different therapeutic applications. First, particles can be delivered to tissues where they act in direct contact with local cells. The activity of such particles then can be used to modify cell proliferation, differentiation or death. Second, peptides, active or inactive macromolecules (including proteins, lipids, carbohydrates, nucleic acids or combinations of these), or entire cells or virus can be adsorbed by these particles and used as a vaccine to enhance the immune response Third, active drugs, agents, proteins or whole cells can be adsorbed within the pores of such particles for delayed delivery to tissues and organs as the rest of their cells are slowly released from pores/cages.

Examples of such bioactive particles are zeolite encapsulated or clay and double hydroxide intercalated metal porphyrin, phthalocyanine, corrinoid and Schiff-base complexes. These can be used as catalytic prooxidants or antioxidants and can modify gene expression regulation and cell fate (proliferation, death or differentiation). Examples of the use of such particles as vaccine adjuvant are mixtures of cancer cells with zeolite particles for enhancing the immunogeneity of cancer cells. Examples of the use of such systems for delayed drug delivery are silica gels, encapsulated catalytic antioxidants or whole cell vaccines. The surface of such particles can be modified by, for instance, adsorption of vitamin B12, for enhanced oral or transdermal adsorption. Particles can also be incorporated into liposomes.

The various features of novelty that characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### DETAILED DESCRIPTION OF THE PRESENTLY PREFERRED EMBODIMENTS

As described in the Introduction, insoluble particles such as silica, talc or zeolites have been used for external cosmetic and therapeutic treatments such baby rash [U.S. Pat. No. 3,935,363]; antimicrobial external treatment [U.S. Pat. No. 5,900,258] or stomach discomfort and enteritis [G. Rodriguez Fuentes et al., Zeolites, Vol. 19, pp. 441-448 (1997)]. Unfortunately, powders could not be used for internal therapeutic applications due to poor adsorption of large micron sized powders. Also, in the prior art, the natural “as obtained” activities of powders were relied upon of activity.

We describe compositions containing submicron and nanosized powders which are nanoengineered to obtain desired therapeutic activity and bioavailability. We also describe methods to prepare such powders and use them independently or with other therapeutic agents. Our approach is biomimetic: we use knowledge on the mechanism of biological processes to produce therapeutic agents that imitate nature's own solutions. It is desirable to produce powders with the maximum therapeutic efficiency and minimum side effects.

The most active powders and colloids commonly contain silicon. Silicas, silicates, clays, double hydroxides and zeolites are examples of these solids. Such solids can be natural or synthetic. Also, such solids can be amorphous or crystalline. These powders can contain only silicon or other nonoxygen-hydrogen components including aluminum, titanium, zinc, iron or silver. Such metals can be part of the crystal structure or encapsulated inside pores. Such powders can be spherically shaped, irregularly shaped, plate-like shaped or fibrous-shaped. Particle size can range from several millimeters to several nanometers. Pore size of such powders can also vary from one tenth of a nanometer to one hundred nanometers. Pore shape can also vary (spherical, cylindrical, spiral etc.). Particle charge can also vary from highly positive to highly negative. The nature of particle wettability (hydrophilic or hydrophobic) can also vary.

The mean particle size of activated silicate/zeolite particles was determined with standard electron microscopy techniques (scanning and transmission electron microscopy), well known to the engineering and scientific community. Electron microscopy is also used to show the absence of fibrous silicates that are considered toxic and interfere with particle size measurements.

In addition, mean particle size was determined with laser light scattering and photon correlation spectroscopy techniques. For example, Malvern Zeta Sizer 3.0 and UPA small particle analyzers were used to determine mean particle size of the above described silicate/zeolite samples. Suspensions with 10 mg/100 ml and pH of 5.5+−0.3 were prepared for that purpose. Suspensions were treated for 5 minutes or more on the ultrasound bath to break any agglomerates.

The preferred average particle size for bioactive silicate solids is about 6 microns or less, preferably about 0.5 to 5 microns, and more preferably about 1.5 microns. Samples contained particles which varied in size from 200 nm to 12 microns. Particles larger than 5 microns can be removed by preparing 1 g/100 ml suspensions and subsequent 1 hour sedimentation. Most particles were of round irregular shape with rough surfaces produced by high energy grinding.

Electrophoretic mobility measurements of suspensions containing 50 mg/100 ml particles at pH of 5.5 or above showed that particles were negatively charged. Electrophoretic mobilities were measured with Malvern Zeta Sizer 3.0 or Zeta Meter 3. Those skilled in the art are familiar with means to measure particle size and charge. Powder X ray diffraction measurements on Scintag or Philipps systems also identified that no amorphization occurred during high energy grinding of crystalline samples such as clinoptilolite zeolite or quartz.

In our approach, nanoengineering is used to prepare powders with desired properties. Only a few examples of preparation will be described in detail. It will be obvious to those skilled in the art how to prepare particles with different properties by using such principles/ideas and referenced literature. For instance, the synthesis of porous materials is described in great detail in such publications as: “Synthesis of Porous Materials, Zeolites, Clays and Nanostructures, eds. M. L. Occelli and H. Kessler; Marcel Dekker, New York. (1997). Journals such as “Zeolites” also deal with similar topics. An excellent review of sol-gel synthetic methods is presented in Brinker and Scherer, “Sol-Gel Science,” Academic Press, San Diego, Calif. (1990). The chemistry of silica and silicate based materials is well described in R. Iler, “Chemistry of Silica,” Wiley, New York, (1979). A recent review of aqueous silicate synthetic chemistry with numerous references appeared in J. Sefcik and A. V. McCormick, AIChE J., Vol. 43, pp. 2773-2783 (1997). Good reviews on encapsulation of metal complexes inside biomimetic silicate catalysts appeared in P. C. H. Mitchell, Chemistry & Industry, May 6, (1991), pp. 308-311; and F. Bedioui, Coordination Chemistry Review, Vol. 144, pp. 39-68 (1995). A good review of the literature on the synthesis of catalytic metal complexes can be found in U.S. Pat. No. 5,834,509 (1998). Many other sources are available on synthesis of functional silicate materials and are well known to those skilled in the art. Many natural and synthetic silicas and zeolites are available from various sources, which will be well known to the skilled in the art (such as Union Carbide, W R Grace, Mobil, Exxon, Akzo, etc.). Only our modifications of such powders will be described.

In prior art, large particles (several microns to several hundred microns) were used for external skin treatment or internal GI tract treatment. In this invention, we describe the synthesis and use of submicron and nanosized powders that are nanoengineered for maximum therapeutic and prophylactic efficiency and for minimal side effects. There are generally three different approaches to producing nanosized silicate particles: 1) high energy ball milling; 2) hydrothermal aqueous synthesis; and 3) sol-gel synthesis. Depending on the precursors used and conditions of the synthesis, various materials such as amorphous silica, clays, double hydroxides or zeolites can be synthesized. Metal complexes or other active molecules can then be encapsulated during or after synthesis. Surface modification or adsorption of active molecules on the particle surface is usually achieved as the last step. Dealumination of zeolites and other modifications of crystal structure or pore chemistry can also be performed. Submicron or nanosized silicate based particles with catalytic entities encapsulated inside the pores and surface modifications are the final products of synthesis. Such particles can then be used alone or with other bioactive substances as a therapeutic or prophylactic product.

Some examples of the preparation of biomimetic catalytic therapeutic solids will be described here. As indicated before, submicron and nanoparticles are more bioactive due to the enhanced transport properties of such materials, particularly in oral and subcutaneous delivery. High energy ball milling, hydrothermal aqueous synthesis and sol-gel synthesis can be used to prepare these small particles.

Zeolites are aluminosilicates with open framework structures constructed from SiO4 and AlO4 tetrahedra linked together through oxygen bridges. Each oxygen atom is shared by two silicon or aluminum atoms. The large variety of zeolites structure types is a consequence of the flexibility of the Al—O—Si linkage, which depends on the conditions during synthesis or natural geological formation. The tetrahedral coordination of Si—O and Al—O permits a variety of ringed structures containing 4, 5, 6, 10 or 12 Si or Al atoms. These rings are joined to form prisms and more complex cages, and the cages are joined to give three, two or one-dimensional frameworks. Because these structures contain uniformly formed sized pores and channels in the range of 4 to 13 Angstroms, zeolites are able to recognize, discriminate and organize molecules with precision that can discriminate for molecular sizes than 1 Angstrom. For example, in natural zeolite faujasite and synthetic counterpart zeolite Y, a supercage of 13 Angstrom is connected via 12 rings of 8 Angstrom to four other cages in a tetrahedral arrangements. During their hydrothermal or geologic synthesis, the channel networks of zeolites are filled with water, which can be removed by heating.

Catalytic metal complexes that we wish to encapsulate into zeolites have quite a large size (7 to 14 Angstroms) and cannot be fixed within zeolite pores by simple ion exchange processes. The so called “ship in a bottle” zeolite based catalysts have to be synthesized with different methods and synthetic strategies, as described below.

### EXAMPLE I

### Flexible Ligand Diffusion+High Energy Grinding to Prepare Catalytic Zeolite Encapsulated Metal Complexes

In a flexible ligand approach, a flexible ligand must be able to diffuse freely through the zeolite pores, but, upon complexation with a previously exchanged metal ion, the complex becomes too large and rigid to escape the cages. This approach is well adapted for zeolite encapsulation of metal-salen complexes [salen=N,N′, bis (salicylaldehyde)ethylendiimine)] since salen ligands offer the desired flexibility. Catalytic salen-metal antioxidants and their synthesis have been described in great detail in U.S. Pat. No. 5,834,509 (1998). Thus, a large variety [N. Herron, Inorg. Chem., Vol. 25, p. 4714 91 986); C. Bowers and P. K. Dutta, J. Catal., Vol. 122, p. 271 (1990); L. Gaillon et al., J. Electroanal. Chem., Vol. 345 p. 157 (1993); K. J. Balkus et al., Zeolites, Vol. 10, p. 722 (1990); S. Kowalak et al., J. Chem. Soc. Chem. Commun., p. 57 (1991)]. of cobalt, manganese, iron, rhodium and palladium salen -metal complexes can be prepared within the zeolite Y or natural faujasite supercages. The synthesis of such complexes encapsulated within zeolite cages described in detail in these references.

In a typical experiment, the appropriate metal cation is placed into zeolite Y supercages (zeolite Y or faujasite can be obtained from various sources such as Union Carbide Corporation) by ion exchange. This can be achieved by heating 5.0 gram zeolite powder suspended in distilled water with 0.05 M metal nitrate for 24 hours at 80° C. filtering, drying under vacuum at 150° C. for 12 hours and subsequent cooling to room temperature. Then, approximately 2.0 g of previously metal exchanged zeolite Y powder is combined with 2.0 g of freshly recrystallized salen and heated to 150° C. Upon fusion, the obtained slurry is stirred for 2-4 hours. The mixture is then cooled to solidify and crushed to a fine powder. The powder is extracted with successive portions of acetone, acetonitrile, dichloromethane and acetone for at least 24 hours each to remove unreacted salen ligand and the surface adsorbed complexes. Such encapsulation results in up to 90% efficiency of metal complex encapsulation. Metaloporphyrins, phthalocyanines and corrinoids can be encapsulated in a similar way.

The powder (1.0 g at a time) obtained is then placed in a planetary high-energy ball mill (Fritsch Pulverisette type 05002) and ground at 3000 rpm in an agate vessel containing about 10 wolfram carbide or zirconia balls (about 10 mm in diameter) for a predetermined time. The best results are obtained by about 10 minutes of grinding. A mean particle size of some 500 nm, with some nanosized particles is achieved without substantial amorphization of the zeolite powder. Longer grinding inevitably results in amorphization and destruction of zeolite supercages. Alternatively, attrition milling or high pressure roll milling can be used but it is difficult to obtain nanoparticles with such milling.

Prepared fine powder is then suspended in distilled water at 1 g/100 ml and 100 mg of vitamin B12 (cyanocobalamin) is added. The mixture is stirred for 2 hours and then filtered through a 0.1 micron filter. This results in significant adsorption of cyanocobalamin at the surface of the zeolite. Recently it was shown that submicron and nanoparticles with the adsorbed vitamin B12 are adsorbed by cells and tissues more efficiently. [G. J. Russel-Jones et al., Int. J. Pharm., Vol. 179, pp. 247-255 (1999)]

### EXAMPLE II

### Template Based Hydrothermal Zeolite Synthesis: Metal Complexes Used as a Template

In hydrothermal synthesis of zeolite materials, one customarily uses organic templates to achieve more efficient synthesis, the desired pore size and crystal structure of synthesized zeolites. Silicate ions are a source of silica. Silicates are customarily prepared by mixing silica with hydroxides to attain the high pH values needed to dissolve silica and prepare silicate ions. Aluminates are used as a source of aluminum (alumina is dissolved with hydroxide). The template is then mixed with silicate and aluminate ions and usually heated at low temperature for a predetermined time. The amorphous product obtained is then filtered, dried and heated at high temperature to crystallize zeolite particles. If desired, the template can then be removed by heating to high temperature (over 300° C.) or by repeated washing with hot alcohol.

Until recently, only metal complexes with neutral molecules were used as templates, which resulted in a very low efficiency of metal complex encapsulation. It was reported that if cationic complexes are used, by analogy to customary zeolite-templated synthesis, much better encapsulation efficiencies are achieved (up to 3%). This is not surprising since silicates are highly negatively charged and are attracted to positive ions.

Metal-salen complexes with cationic charges on salen salycilidene aromatic rings are available. The preparation of metal-salen complexes is described in great detail in U.S. Pat. No. 5,834,509. In general, salycylaldehide with desired substituents and ethylenediamine with desired substituents are mixed in 2:1 ratio in organic solvents, preferably absolute ethanol. The solutions are refluxed, typically for 1 hour, and the salen ligand is precipitated by adding metal acetate or halide in an appropriate amount. The precipitated powder is filtrated and washed with cold ethanol. If one starts with salcylaldehide substituted with cationic, tetramethyl alkyl species, such as is described in [S. Bhattacharya and S. S. Mandal, J. Chem. Soc. Chem. Commun., p. 2489 (1995)], one produces bis cationic salen complex. In the chosen example, salycylaldehide had substitution at the third carbon atom. [S. Bhattacharya and S. S. Mandal, J. Chem. Soc. Chem. Commun., p. 2489 (1995); FIG. 1b] The substituted carbon chain was R═O(CH2)3—NMe3+. Other cationic substitutions are possible. The metal ion used in this particular case was cobalt (II).

Starting with silicate, aluminate and such cationic templates, standard procedures can be applied to obtain zeolite with larger pores (typically synthetic zeolite Y). In one typical synthesis, 300 mg of cationic salen-cobalt complex, described in [S. Bhattacharya and S. S. Mandal, J. Chem. Soc. Chem. Commun., p. 2489 (1995)], was added to freshly prepared aluminosilicate gel. The gel was prepared by mixing 4.6 g of silica, 6.2 g of NaOH and 3.2 g of NaAlO₂ and 80 ml of water. The gel was then crystallized at 95° C. under static conditions in a stainless steel bomb (250 ml) for 48 hours. After cooling to room temperature, a solid crystalline product was recovered by filtration. The complexes adsorbed on the exterior surfaces were removed by a thorough extraction with distilled water, methanol, pyridine, and methanol again, respectively. The crystals were then dried at 60° C. for 12 hours.

Prepared fine powder is then suspended in distilled water at 1 g/100 ml and 100 mg of vitamin B12 (cyanocobalamin) is added. The mixture is stirred for 2 hours and then filtered through a 0.1 micron filter. This results in significant adsorption of cyanocobalamin at the surface of the zeolite. It was shown that submicron and nanoparticles with the adsorbed vitamin B12 is absorbed inside cells much more efficiently. The average particle size of the so obtained zeolite was 300 nm with up to 25% nanoparticles.

### EXAMPLE III

### Template Based Hydrothermal Alumina Free (the So Called Silicalite) Zeolite Synthesis: Cationic Metal Complexes Used as a Template

It has been postulated that long term use of solids containing aluminum might be toxic due to the aluminum content. Therefore, it is also advantageous to synthesize aluminum free zeolites with catalytic templates. A similar approach to that described in Example II, but without the addition of any aluminate ions, is used.

For instance, 1 gram of cationic cobalt-salen complex, described in [S. Bhattacharya and S. S. Mandal, J. Chem. Soc. Chem. Commun., p. 2489 (1995)] was added to a gel produced by addition of 5.0 g of silica to 10 g of tetrapropylammonium hydroxide. Approximately 10 g of water was added to this gel. The resulting homogeneous viscous mixture was left standing for 24 hours and then placed in a stainless steel bomb and heated at 50° C. for 14 days. The resulting crystalline solid was filtered and dried at 60° C. overnight. If needed, tetrapropylammonium ions can be removed from pores by boiling in ethanol for 24 hours. The Cobalt-salen complex is larger than the pore size and is not removed with this treatment.

The resulting zeolite encapsulated metal complexes have to be analyzed to ensure that the desired products are obtained. X-ray diffraction and FTIR analysis are used to check that crystalline and not amorphous materials are obtained. Chemical analysis, X-ray fluorescence and X-ray photoelectron spectroscopy are used to determine chemical compositions of the obtained products. Thermal gravimetric analysis can be used to analyze the stability of the obtained products. High-resolution transmission electron microscopy can be used to obtain information about the zeolite crystalline structure on the nanoscopic level. TEM and SEM can also be used to obtain information about particle size and shape. Electrophoretic mobility measurements can be used to determine particle charge.

In general, small submicron or nanosized particles with a crystalline rather than amorphous form are desired. Irregularly shaped particles are better adsorbed by the body. Fibers are considered potentially toxic and should be avoided. Negatively charged particles are usually desired, positively charged particles can adsorb to DNA and break it, resulting in mutations. High adsorption of surface modulating agents such as vitamin B12 are desired (to enhance bioavailability). High concentration of encapsulated metal complexes are desired (at least 1% of pores should be filled with catalytic metal complexes).

It is postulated that that zeolites with high percentages of aluminum are toxic, It is, however, easy to remove aluminum from the zeolite framework without the loss of catalytic ability. Several US patents describe different ways in which dealumination can be achieved. For instance, U.S. Pat. No. 5,900,258 describes a very efficient way to dealuminate zeolites by acid HCl leaching. Dealumination can also be achieved with milder weaker acids (methane sulfonic acid, for instance) as it is described in U.S. Pat. No. 5,508,019. Literally hundreds of other successful dealumination techniques are described in the literature which would be familiar with those skilled in the art.

In the prior examples, zeolite encapsulated metal complexes were synthesized for their catalytic activity as antioxidants or prooxidants. Biomimetic solids can also be used as vaccine adjuvants and delayed active pharmaceutical products delivery reservoirs. Different features are desired for such biomimetic solids. The preparation of some systems designed for such use will be described below.

The biomimetic solids that can be used as delayed active pharmaceutical agent delivery reservoirs must have larger pores so that larger reagents such as proteins or whole cells can be incorporated when desired. Also, the affinity of such solids for the encapsulated ingredients should not be too strong because of the possibility of irreversible encapsulation. Excellent micro, meso and macro-porous aluminosilicates and silicas have been synthesized in recent years. For our purposes, such systems have to be milled to obtain smaller particles. The surface of the particle has to be modified for enhanced adsorption into tissue and organs. Pores should be modified in order to release encapsulated active ingredients with the desired kinetics. Since such particles are commonly used for oral or mucosal delivery, they should be dealuminated to avoid aluminum dissolution in the stomach and possible toxicity. Only a few examples of such modifications will be described. Those skilled in the art will be able to use such examples and the text of this patent to design other possible modifications that are also included in this patent.

Mesoporous aluminosilicate with pore size up to 2 nm have been prepared by Mobil Corporation researchers [U.S. Pat. No. 5,211,934]. Such crystalline aluminosilicates have very high adsorption capacity. The pore size of such particles is large enough to adsorb and slow release most common small molecule drugs and even small proteins such as insulin. Such particles can be dealuminated by leaching with 6 N HCl as described in U.S. Pat. No. 5,900,258. Dealumination can increase silica alumina ratio up to 250:1. Grinding in a high-energy ball mill or attrition mill with zirconia balls can then reduce particle size to the desired value (submicron and nanoparticles are preferred). Mixing with the desired small molecule pharmaceutical agents can then result in strong adsorption (up to 30 g of adsorbed molecules per 100 g of aluminosilicate). The surface of the aluminosilicate particles can then be modified with the adsorption of, for instance, vitamin B12, in order to enhance bioavailability, as described earlier.

Another logical choice for a biomimetic solid with a variety of pore sizes and the ability to modify the pore and surface chemistry, is silica particles. Numerous manufacturers offer a large variety of different silica samples. Silica gel particles are, for instance, manufactured by W. R. Grace & Co., Davison Chemical Division (SyloidR silicas). Such particles have surface areas from about 250 to 400 m²/g and average particle size of 2.5 to 6 microns. Average pore size can be as large as 100 nm. Fumed silica particles are much smaller with mean particle size from 6 nm to 30 nm. Such samples can be obtained from, among others, Cabot Corporation, Tuscolla, Ill. (Cab-OSilR series). DuPont Corporation or Nissan Corporation also sells a large variety of silica samples. Such particles, obviously, do not have to be dealuminated. Since silicas are already amorphous, high energy grinding for particle size reduction cannot have detrimental effects on particle activity. Such particles are generally also cheaper than aluminosilicates. Silica particles contain a large number of surface and pore hydroxyl groups and can, therefore, easily be modified with many different molecules, such as silane coupling agents. Virtually any desired particle size, pore size and wettability are commercially available. The challenges of biomimetic synthesis are to modify the surface of silica particles to achieve maximum bioavailability and to modify pore chemistry in order to achieve slow delayed release kinetic of the adsorbed active ingredients. Some examples of preparing such biomimetic silicas will be described when pharmaceutical activities are discussed below. In general, active ingredients are either mixed at room temperature or refluxed in water or ethanol with silica particles in order to achieve the desired adsorption/absorption. The surface of the silica particles can then be modified, either by chemabsorption or physical adsorption of desired molecules needed to increase particle bioavailability. The previously described approach, with the adsorption of vitamin B12 on the surface, is again applicable.

The third area of application of biomimetic solids is their use as vaccine adjuvants in order to enhance the immunogeneity of various vaccines. It is well known to those skilled in the art that most proteins and even bacterial cells or tumor cells are poorly immunogenic when used alone. Some additional materials have to be used as adjuvants to enhance the vaccine's immunogeneity. [D. L. Morton in Cancer Medicine, Vol. 1; eds. J. F. Holland et al., Williams and Wilkins, Baltimore (1997), pp. 1169-1199] A large number of recent publications report that polymer particles can enhance the efficiency of many vaccines. We will describe the use of crystalline zeolite particles such as natural clinoptilolite or fumed silica particles to enhance the immunogeneity of tumor cells and bacteria. High energy grinding produces small particles that are active vaccine adjuvants. Zeolite and silica particles with rough edges and irregular shapes penetrate inside cell membranes and modify the ordering of surface proteins, making them more immunogenic. The preparation of such vaccines is simple: after grinding and eventual surface modification of zeolite particles, one mixes a predetermined amount with vaccine cells and prepares a standard solution for subcutaneous or even oral delivery of such vaccine. If zeolites are prepared to act as catalytic oxidants, this attracts even more macrophages and other lymphocytes. It is well known that oxidative free radicals are attractant for macrophages and other lymphocytes.

Another way to enhance a whole cell vaccine is to incorporate whole living cells inside silica gel. Such gels can be prepared by acidification of sodium or potassium silicates in a similar way as silicalite synthesis described in example III. Whole cells are encapsulated inside silica gel and are also modified to use their ability to divide. Therefore, one can use live cells, which is the best Way to deliver vaccine. [D. L. Morton in Cancer Medicine, Vol. 1; eds. J. F. Holland et al., Williams and Wilkins, Baltimore (1997), pp. 1169-1199] Since whole cells are diffusing very slowly out of the gel, one vaccine applications might be enough for weeks or even months of immunity. The viscosity of such gels can be adjusted so that the gel can be filled into a syringe and used for subcutaneous delivery of the vaccine. As in the case of zeolite, the surface of the gel can be modified, for example by adsorption of vitamin B12, for better bioavailability. Catalytic salen-cobalt prooxidant complexes can be incorporated inside pores to produce superoxide radicals [S. Bhattacharya and S. S. Mandal, J. Chem. Soc. Chem. Commun., p. 2489 (1995)] which are known to be attractant for macrophages and other lymphocytes. Cytokine protein such as IL-12 or GM-CSF can also be added to silica gel. Such peptides further assist in the enhancement of the immune response towards cancer cells. Those skilled in the art are familiar with many different ways to synthesize silica gels and vaccines enhanced in such way are therefore included in this patent. Those skilled in the art will be able to easily design a large variety of modifications of such Vaccines enhancing silicas and these modifications are, therefore, encompassed by this patent.

### Biological and Therapeutic Activities of Biomimetic Solids

This invention describes three different uses of biomimetic solids. First, biomimetic solids can be engineered to become catalytic pro-oxidants or antioxidants and modify gene expression and tissue/cell behavior upon direct contact. This will result in changes in cell proliferation, growth, differentiation or death. Such catalytic effects are possible only in direct contact with tissue/cells and biomimetic solids are engineered for enhanced internal transport. Such activities will then be engineered to help cure or prevent different disease conditions. Second, biomimetic solid particles can be used as vaccine adjuvants to enhance the immunogeneity of proteins, cell parts or whole cell vaccines. Third, biomimetic solids and gels can be used to incorporate small drugs, cosmetic agents, macromolecules or whole cells for a slow delayed sustained release. Some particular results and examples of the biological and therapeutic activities of biomimetic solids are described below.

### EXAMPLE IV

### Antioxidants and the Anticancer Activity of Biomimetic Zeolite

It was recently observed by numerous researchers that natural and herbal antioxidants can stop the uncontrolled growth of some cancer cells and even enhance the anticancer activity of chemotherapy agents. Many patients claim that eating food rich in plants and fruits, soybeans, polyphenol sources such as green tea and even powdered zeolites helped in their fight against cancer. Some of the most legitimate stories come from patients suffering from adenocarcinoma of the lung, breast or colorectal adenocarcinoma. Some success has also been reported with melanoma and glioblastoma treatment.

What is the biochemical mechanism of action of such a diverse group of products as soybeans, green tea and zeolites? While we do not wish to be bound by any mechanism of action, the following is a reasonable possibility. The common activity noted with most of such dietetic products is that they act as potent antioxidants and free radical scavengers. In recent issues of Methods of Enzymology (Vol. 299, 300 and 301) it was clearly shown that dietetic products indeed outperform vitamin C, E and other classics of antioxidants by more than an order of magnitude in their ability to scavenge free radicals and produce a more reducing environment inside cells. The question now is how can potent antioxidants influence cell proliferation, differentiation and death? Scientists have just started to understand the underlying mechanisms. Chinnery and coworkers reported in Nature Medicine, Vol. 3, pp. 1233-1241 that strong antioxidants such as pyrrollidinedithiocarbamate and N-acetyl cysteine caused partial remission in-vitro and in-vivo when added to colorectal adenocarcinoma in tissue culture and when fed to mice with implanted tumors. Moreover, when used with chemotherapy agents such as 5-fluorouracil or adriamicin, antioxidants enhanced the cytotoxicity of chemotherapy agents and caused complete remissions where only partial remission was possible with the chemotherapy agent only.

Chinery and coworkers went one step further and asked the question: why did this happen? Recent studies indicated that some of the most potent molecules that control cell growth and possible tumorigenesis are tumor suppressor molecules. Such molecules modify gene expression and the activity of proteins involved in the initiation of cell division. Cyclins were identified as molecules which directly stimulate cell division. On the other hand, cyclin kinases are needed to activate cyclin molecules by phosphorilation, a common signal transduction strategy. Some of the most potent tumor suppressor molecules are actually inhibitors of cycline kinases CDK-2 and CDK-4. Two of these molecules are known as p21/WAF1/CIP1 and p27/KIP1. Another common tumor suppressor molecule p53 is actually needed to activate p21/WAF1/CIP1.

Chinnery and coworkers showed that antioxidants induce transcription of p21/WAF1/CIP1 without the need for p53, which is actually inactivated in almost half of human tumors. They further showed that the transcription factor which activates the transcription of p21 gene is actually C/EBP, also known as NF-IL6. They went even further and showed [J. Biol. Chem., Vol. 272, pp. 30356-30361 (1997)] that C/EBP in its activated form actually moves from cytoplasm to nucleus where it stimulates transcription of p21/WAF1/CIP1 by binding to the CCAAT enhancer sequence of DNA. Chinnery and coworkers also identified the possible first step in the activation of p21/WAF1/CIP1. That is antioxidants reduced protein kinase A activity. A reduced form of protein kinase A binds to the membrane, becomes activated and phosphorilates C/EBP, which causes its translocation to the nucleus.

A whole series of papers on anticancer activity of dietetic products showed a similar mechanism of action. Bai and coworkers in Kyoto showed [F. Bai et al., FEBS Lett, Vol. 437, pp. 61-64 (1998)] that plant flavonoids induced p21/WAF1/CIP1 in A549 human lung adenocarcinoma cells. This resulted in growth arrest and apoptosis. The growth arrest was independent of p53. Kuzumaki and coworkers [T. Kuzumaki et al., BBRC, Vol. 251, pp. 291-295 (1998)] showed that genistein from soybeans also induces p21/WAF1/CIP1 and blocks the G1 to S phase transition in mouse fibroblast and melanoma cells. Sadzuka and coworkers showed that green tea extract enhanced chemotherapy activity of adriamicin, in vitro and in-vivo towards ovarian cell cancer with low sensitivity to adriamicin [Clinical Cancer Research, Vol. 4, pp. 153-156, (1998)]. Nakano and coworkers showed that butyrate activated p21/WAF1/CIP1 in p53 independent manner in human colorectal cancer cell line. This also resulted in growth arrest [K. Nakano et al., J. Biological Chemistry, Vol. 272, pp. 22199-22206 (1997)]

Yet it seems that there are also other similar mechanisms of inhibition of cyclin and retinoblastoma protein phosphorylation. Frey and coworkers showed that agonists of protein kinase C alpha isozyme activated both p21/WAF1/CIP and p27/KIP1 tumor suppressors. This resulted in growth arrest and hypophosphorilation of both cyclin molecules and retinoblastoma protein, which is also involved in carcinogenesis. [M. R. Frey et al., J. Biological Chemistry, Vol. 272, pp. 9424-9435 (1997)]

Carlson and coworkers at National Cancer Institute in Bethesda and their coworkers from Mitotix corporation identified a flavonoid which actually directly bound to CDK-2 and CDK-4 and inhibited both of these cyclin dependent kinases directly. [B. A. Carlson et al., Cancer Research, Vol. 56, pp. 2973-2978 (1996)] This resulted in growth arrest of human breast carcinoma cell line. S. H. Kim and coworkers from UC Berkeley determined even the 3D structure of the complex between CDK-2 and such flavonoid. This data will be very useful for the future design of more potent cyclin dependent kinase inhibitors. [W. Filgueira et al., PNAS, Vol. 93, pp. 2735-27740 (1996)]

Based on these results, we speculated that if powerful catalytic antioxidants are delivered to cancer cells, they could even more efficiently stop their uncontrolled growth. Catalytic antioxidants can scavenge large number of oxidants before they are themselves inactived. All other natural and herbal antioxidants are stoichiometric antioxidants, meaning that they can act only in a 1:1 ratio, so they are used quickly, limiting their use.

Zeolite encapsulated catalytic antioxidants have another advantage in that encaged molecules cannot get in direct touch with each other and loose activity through multimerization. Also, they cannot react or bind to macromolecules and loose activity in such fashion.

In this example, we used manganese-salen complex described in U.S. Pat. No. 5,834,509 (1998) and K. Baker et al., J. Pharmacol. and Exp. Therap., Vol. 284, pp. 214-221 (1998). The process described in EXAMPLE I was used to encapsulate manganese-salen complex inside zeolite Y/faujasite cages. Such powder was then used to follow its anticancer activity in in-vitro tissue culture and in-vivo nude mice with implanted tumor experiments.

Cell/tissue culture experiments: several different human and mouse cell lines, such as lung adenocarcinoma, colorectal adenocarcinoma, breast adenocarcinoma, melanoma and glioblastoma were investigated. Various amounts of zeolite encapsulated salen-manganese complex were used. The maximum growth arrest of cancer cell lines was achieved at 50 mg/ml of added zeolite. In all cases studied this amount of zeolite caused complete growth arrest of cancer cells.

In one experiment, human A549 lung adenocarcinoma cells are cultured in Dulbecco's modified Eagle's medium (DMEM) (Sigma Chemicals, St. Louis) containing 10% fetal bovine serum and grown at 37° C. in a humidified atmosphere of 5% CO2 in air. A549 cells were seeded at a density of 1×104 cells/2ml of medium in 35 mm diameter dishes. Various amounts of zeolite, 0.1-50 mg/ml) were added to cells 24 hours after seeding. Twentyfour, 48 and 72 hours after the addition of zeolite, the number of live cells was determined by the Trypan blue dye exclusion test. This cell growth test was carried out in triplicate and repeated at least three times. Complete growth arrest of cancer cells was achieved only at the highest concentrations of zeolite used.

To show that strong antioxidant activity of zeolite encapsulated catalytic antioxidants correlated with anticancer activity, we measured the ability of zeolite to reduce oxidative damage in cell culture experiments. Intracellular oxidative damage to 1,2,3 dihidrorhodamine (DHR) (Molecular Probes, Eugene, Oreg.) was measured using flow cytometry. Cells (A549) were grown in DMEM containing 1 mM DHR for up to 24 hours. Control cells were grown without the addition of zeolite, and test cells were grown with various amounts of zeolite (0.1-50 mg/ml). Following trypsinization, trypsin activity was quenched with 2% fetal bovine serumin PBS, and cells were fixed in 1% paraformaldehyde. Cellular oxidized 1,2,3 rhodamine fluorescent intensity was measured for each sample (1×104 cells) using FACS with an excitation source of 488 nm and emission wavelength of 580 nm. Histograms were analyzed with the software PC-Lysis (Becton-Dickinson). Background fluorescence from blank wells was subtracted from each reading. Zeolite treatment at the highest dosage could completely abolish rhodamine 1,2,3 production inside cancer cells for up to 24 hours.

In animal tests, male athymic Balbic nu/nu mice were obtained from the Harlan Sprague-Dawley Company (Indianapolis, Ind.) at 4-6 weeks of age and were quarantined for 2 weeks before the study. Animal experiments were carried out in accordance with both institutional and federal animal care regulations.

A549 adenocarcinoma (as well as other cell types mentioned before) were grown in DMEM media supplemented with 10% fetal bovine serum as described above. Cells were harvested through two consecutive trypsinizations, centrifuged at 300 g for 5 min, washed twice, and resuspended in sterile phosphate-buffered saline (PBS). Cells (1×10⁶) in 0.2 ml were injected subcutaneously between the scapula of each mouse. Tumor volumes were estimated weekly by measuring the maximum length, width and height. Once tumors reached a mean size of 150 mm³, the animals received the following treatment: daily admixed zeolite with their food (mice chow) in a 1:3 ratio. It is estimated that animals consumed some 500 mg/kg of zeolite per day. Ten animals received only normal food and another ten animals received zeolite enriched food. After 4 weeks, all control animals had to be sacrificed due to excessive tumor size some even larger than the mouse's normal body. Among treated animals, 3 showed complete remission, 4 partial remissions (up to 70% of the tumor volume of the controls) and three showed similar tumor sizes to the controls. Similar results were observed with colorectal and breast adenocarcinoma models. No complete remissions were ever observed with melanoma tumors.

### EXAMPLE V

### Antidiabetic Effects of Zeolite Encaged Catalytic Antioxidants

The same zeolite sample used in EXAMPLE IV was used in Example V. The antidiabetic effects of such zeolite were tested with diabetes prone NOD mice models.

Twelve female diabetes prone NOD mice were obtained from the Jackson Laboratory. 10 male non-diabetes prone NOD mice were obtained from the same source and used as controls. The mice were obtained at ten weeks of age. Mice were fed mice chow with 50% of admixed zeolite.

Glucose in the blood was measured weekly. At the time of death, lipid oxidation products in serum and pancreas tissue were measured (TBAR's).

Male mice were used as a control. Out of 10 male mice, 8 did not develop any signs of diabetes. The amount of glucose in the blood of such animals was 5.2+−1.45 mmol/l without significant variations.

At 25 weeks of age, the differences in glucose blood levels started to appear: Six female mice were fed normal drinking water. Out of those six, five developed diabetes. At 25 weeks of age, they had 25+−4.2 mmol/l of glucose in the blood.

At 25 weeks of age, five out of the six female mice fed zeolites developed diabetes, but the average glucose in blood was only 8.1+−2.2 mmol/l.

At the time of death (26 weeks of age), the amount of oxidized lipids was determined in all mice. Female mice which developed diabetes and were fed normal water had 320+−35% higher amount of TBAR's in their blood than male mice which did not develop diabetes. Female mice fed zeolite enriched food had 120+−25% higher amount of TBAr's than the male mice which did not develop diabetes. Thus, while this treatment reduced oxidative damage and lowered blood glucose, it did not completely stop the development of diabetes.

### EXAMPLE VI

### Antimicrobial Activity of Pro-Oxidant Catalytic Zeolite

It is well known that oxidants such as hypochlorous acid, hydrogen peroxide, hydroxyl radical and ozone are used by both industry and our body to kill microbes. Recently, it was also recognized that silver and zinc encapsulated within zeolites can enhance their antimicrobial activity. This can be used in skin care, oral care and even for internal infections or wound treatment. However, in prior art only large particles with limited transport and bioavailability were used. In this invention, we describe the preparation of submicron and nanosized antimicrobial zeolites.

First, zeolite encapsulated pro-oxidant cobalt II-salen complex is prepared as described in EXAMPLE II. Ten grams of this powder was then suspended in 200 ml of water. Silver nitrate and zinc chloride was then added to 0.05 M of each salt. The resulting suspension was heated to 80° C. and mixed for 48 hours. Zeolite powder was filtered and dried at 60° C. for 8 hours. The obtained powder (1.0 g at a time) is then placed in a planetary high-energy ball mill (Fritsch Pulverisette type 05002) and ground at 3000 rpm in an agate vessel containing about 10 wolfram carbide or zirconia balls (about 10 mm in diameter) for a predetermined time. The best results are obtained by 10 minutes of grinding. A mean particle size of around 500 nm, with some nanosized particles is achieved without substantial amorphization of the zeolite powder. Longer grinding inevitably results in amorphization and the destruction of zeolite supercages. Alternatively, attrition milling or high-pressure roll milling can be used but it is difficult to obtain any nanoparticles with such milling. Those skilled in the art are familiar with different grinding technologies that can be used. The use of various grinding methods not mentioned herein is, therefore, also incorporated into this patent.

The prepared fine powder is then suspended in distilled water at 1 g/100 ml and 100 mg of vitamin B12 (cyanocobalamin) is added. The mixture is stirred for 2 hours and filtered through a 0.1 micron filter. This results in significant adsorption of cyanocobalamin at the surface of the zeolite. It was recently shown that submicron and nanoparticles with the adsorbed vitamin B12 are absorbed inside cells and tissues much more efficiently.

The prepared powder is then dried at 60° C. for 8 hours and is ready for use. Such powder was tested for its antibactericidal activities with over 20 common different bacteria (E coli, S. aureus, etc.) and yeasts (C. albicans etc.). In most cases, 15 minutes of equilibration with a suspension containing 10 mg/ml of zeolite caused at least a five log decrease in the count of bacteria. This shows great potential to use such powders in oral hygiene, skin care, feminine hygiene and wound treatment. The better bioavailability of such powders enables much more potent effects of such biomimetic powders compared to prior art and it is believed that they can also be used for the treatment of internal infections.

### EXAMPLE VII

### Vaccine Adjuvant Activity of Biomimetic Solids

It is a common practice to add an adjuvant component to enhance the immunogeneity of vaccines. Dead bacteria or parts thereof, with toxic substances removed are commonly used for such applications. Inorganic powders such as aluminum hydroxide are also commonly used. [D. L. Morton in Cancer Medicine, Vol. 1; eds. J. F. Holland et al., Williams and Wilkins, Baltimore (1997), pp. 1169-1199] A large number of recent publications report that nanosized or submicron polymer particles can enhance the efficiency of many vaccines. [see for instance S. Novakovic et al., Int. J. Mol. Med., Vol. 3, pp. 95-102 (1999)] Biomimetic nanoengineered solids are particularly good example of agents that can be intelligently engineered to enhance the immune response from vaccines. To achieve that end, we use ground, natural highly crystalline clinoptilolite from the Anatolia region of Turkey (85% pure, with the other components mostly other aluminosilicates). These ground particles have rough edges and can penetrate successfully inside cells.

The following procedure was used to engineer clinoptilolite particles for maximum immunogeneity:

Ten grams of natural clinoptilolite powder was suspended in 200 ml of water. Silver nitrate and zinc chloride were added to 0.05 M concentration of each salt. The resulting suspension was heated to 80° C. and mixed for 48 hours. Zeolite powder was then filtered and dried at 60° C. for 8 hours. The obtained powder (1.0 g at a time) was placed in a planetary high-energy ball mill (Fritsch Pulverisette type 05002) and ground at 3000 rpm in an agate vessel containing 10 wolfram carbide or zirconia balls (10 mm diameter) for a predetermined time. The best results were obtained by 15 minutes of grinding. A mean particle size of around 250 nm, with some nanosized particles, was achieved without substantial amorphization of the zeolite powder. Longer grinding inevitably resulted in amorphization and destruction of zeolite supercages. Alternatively, attrition milling or high-pressure roll milling can be used but it is difficult to obtain nanoparticles with such milling. Those skilled in the art are familiar with different grinding technologies that can be used. The use of various grinding methods not mentioned herein is, therefore, also incorporated into this patent.

The addition of zinc and silver further helped in attracting lymphocytes and augmenting immune response. In animal experiments using nude mice with implanted melanoma or lung adenocarcinoma, 0.3 ml of suspension containing 10 mg of such zeolite, 1 mg of cyanocobalamin and 1 mg of cysteine were injected subcutaneously near the tumor site. Cyanocobalamin+cystein combination produce reduced cobalt II which attracts oxygen and releases superoxide free radicals, which further attract lymphocytes. [L. G. Rochelle et al., J. Pharmacol. Exp. Therapeutics, Vol. 275, pp. 48-52 (1995)] In another set of mice, 1×10⁶ of the autologous syngeneic melanoma or adenocarcinoma (colorectal or lung) cells which were used to inoculate mice for tumor growth were added to the zeolite suspension and 0.3 ml was injected near the tumor site subcutaneously. Mice were injected weekly for the period of four weeks and then sacrificed. Upon death, histopathological studies of tissue near the injection and tumor tissue were performed with standard H&E paraffin blocks and stains. The tissues were analyzed and graded for infiltration of lymphocytes, macrophages and eosinophils. Tumor size was also evaluated.

Mice that were not treated with zeolite or zeolite+cell vaccine developed large tumors and had to be sacrificed for humane reasons due to large tumors four weeks after the start of experiments. Seven out of the animals injected with zeolite only showed significant infiltration of macrophages, T cells and eosinophils near the injection site and inside tumors. Those animals showed partial regressions of tumors up to 70% in size at the time of death (four weeks after the inoculation). Eight out of ten animals injected with zeolites+melanoma cell vaccine showed a very significant infiltration of macrophages, T cells and eosinophils at the tumor site. Three animals showed a complete remission of tumor growth and four other exhibited very strong partial remission of tumor growth. Similar results were observed with adenocarcinomas of lung and colorectal adenocarcinoma models.

The advantage of our approach is that crystalline zeolites strongly enhance immunogeneity of live cell vaccine. Another significant advantage is that zeolites cause the growth arrest of live cancer cells and therefore live cells can be used as a vaccine. Other authors showed recently that using live vaccine cells is the best way to initiate immune response against tumors. [D. L. Morton in Cancer Medicine, Vol. 1; eds. J. F. Holland et al., Williams and Wilkins, Baltimore (1997), pp. 1169-1199]

In addition to mixing live cells with zeolites, other immunogenic species such as tumor specific antigen proteins or peptides can be mixed with zeolites. In addition to zinc, silver and pro-oxidants metal complexes, other species can be added to zeolites to enhance immune response. Cytokines such as interleukin 12 (IL-12), GM-CSF or interferon gamma can be added. Cells or tumor antigens from many different tumors can be added. This can significantly enhance vaccine efficiency. [D. L. Morton in Cancer Medicine, Vol. 1; eds. J. F. Holland et al., Williams and Wilkins, Baltimore (1997), pp. 1169-1199] To our knowledge, this is the first time that live vaccine cells which were not irradiated could be used effectively for tumor treatment.

A similar approach can also be used with vaccines used against bacteria, viruses and larger parasites. In such applications, preliminary vaccination is usually much more efficient. Those skilled in the art are familiar with necessary modifications of vaccine preparations for different organisms (viruses, bacteria etc.) and such modifications of the general strategy used here are included in this patent.

### EXAMPLE VIII

### Delayed Sustained Release of Small Molecules, Macromolecules or Cells Encapsulated Within Biomimetic Solids (Either Within the Particle Pores or in the Interparticle Space)

Active components in our cells, tissues and organs, such as hormones, cytokines or growth factors are released as needed generally in a sustained manner. When a disease state occurs, drugs are often administered as a one-time bolus dose. While this is satisfactory in some cases, sustained release of pharmaceutically active agents would be much more advantageous for most therapeutic applications. Biomimetic solids are an ideal reactor/reservoir for such delivery. Since zeolites, mesoporous aluminosilicates and silicas are available with pores ranging from 1 Angstrom to 100 nanometers, virtually any kind of pharmaceutical agents can be incorporated and later slowly released. Pores can also be modified, so that they have a certain shape, wettability and charge which would modify the rate of pharmaceutically active agent release. Dealumination will generally yield aluminosilicates with larger and more hydrophobic pores. Treatment with methanol or silanes can also hydrophobize pores, as described in U.S. Pat. No. 5,013,700. Cationic, anionic, zwitterionic or nonionic surfactants and silanes can also be used to modify pore charge, wettability and size. Simple mixing of appropriate reagent with zeolite or silica in ethanol or water is usually enough to achieve needed modifications. Particles can later be filtered, dried and resuspended in a suitable solvent such as water or DMSO for pharmaceutical delivery. Particles can be milled to achieve required particle size for maximum bioavailability. Particle surface can also be modified to enhance bioavailability. This can be achieved in a similar way as the treatment of pores. Those skilled in the art are familiar with chemical treatments needed to modify silica based materials and will be able to prepare many such solids with modified pore chemistry or surface chemistry. Such solids are therefore included in this patent. A large variety of surfactants are available from Sigma Chemicals, St. Louis, Mo. Gelest, of Tullytown, Pa. manufactures a large number of silanes and provides excellent technical advice to those wishing to use such chemistry to modify silica and silicate based solids.

A few examples of use of silica based biomimetic solids for delayed sustained delivery of pharmaceutically active agents will be described below.

Both natural and synthetic zeolites clinoptilolite and mordenite have very small pores suitable for delayed release of metal ions. They can be used for the delayed use of silver and zinc, which augment the immune system and also have antimicrobial activity of their own. Simple mixing of 0.05 M of silver nitrate and 0.05 M of zinc nitrate with either powder results in ion exchange. Heating to 70° C. during mixing enhances ion exchange. After 24 hours of equilibration, zeolite powder can then be filtered, dried and ground in high-energy ball mill described earlier, for instance in EXAMPLE I. Such fine crystalline powder can also be mixed with herb echinacea, (1:1) ratio, to further enhance augmentation of the host's immune system. Powder can be applied externally for the skin or wound treatment or can be packed into capsules and taken orally. A combination of external use of such powders on the skin surface and internal intake (twice a day 1000 mg) resulted in significant improvement in 8 out of 10 acne patients. Significant improvements were also observed with 12 out of 16 diabetes patients who had nonhealable open wounds. Once again, a combination of internal and external use was applied. Zeolite powders described in prior art could not achieve such efficiency, probably due to large particle size used in such applications.

As mentioned in the EXAMPLE I, catalytic manganese-salen antioxidants are excellent therapeutic agent for many uses where it is desirable to modify redox controlled gene expression, for example, in cancer treatment. A major problem with most antioxidants is that they are cleared quickly from the body. A problem with use of zeolite described in EXAMPLE I is that, even though the particles are very small, they cannot penetrate everywhere needed. When aluminosilicates or silicas with larger particles are used, such metal-salen complexes are no longer trapped like the “ship in the bottle” complexes described in the EXAMPLE I. Therefore, such molecules are slowly released and delivered to the tissue desired. Mobil Corporation manufactures novel type of aluminosilicates with pores as large as 2 nm, which are ideal for such applications. [U.S. Pat. No. 5,211,934] Metal-salen complexes can be adsorbed inside the pores by heating and refluxing with aluminosilicate powders suspended in ethanol. After 24 hours of refluxing, particles should be filtered, dried and ground in a high-energy ball mill to prepare samples with submicron or nanosized particles.

Finally, large protein or DNA macromolecules can be adsorbed into silica gel pores by mixing in potassium buffered saline (PBS). As indicated, pores can be modified in order to achieve the desired release rate. Cyanocobalamine (vitamin B12) can be adsorbed on the surface to enhance particle uptake and bioavailability. A particular advantage of this approach is that all particles which are adsorbed orally through Peyers patches in the GI tract can deliver protein molecules into the blood without degradation by stomach acid and enzymes.

If whole cells or tissue samples are to be incorporated into biomimetic solids, one can admix them with the freshly prepared silica gel (prepared by acidification of silicates or hydrolysis of tetrathylorthosilicate) in PBS. Gel can be injected subcutaneously as a vaccine or used surgically during artificial tissue or organ implantation, as it becomes possible in the future. A detailed description of numerous synthetic routes to prepare silica gel can be found in [R. Iler, “Chemistry of Silica,” Wiley, New York, (1979)]

As shown in the previous examples, biomimetic solids can be used alone or with other pharmaceutically active ingredients. Biomimetic solids can be applied orally, topically, subcutaneously, intraperitoneally or intramuscularly. Those skilled in the art are familiar with the procedures for preparations of pharmaceutically acceptable products. Numerous literature sources on the subject are available and well known to those skilled in the art. [Remington's Pharmaceutical Science, 15 th Ed. Mack Publishing Company, Easton, Pa. (1980)] Typical dosages of biomimetic solids should be determined in clinical trials and through the interaction of patients and physician. Usually, between 500 mg and 15 gram per day are needed. Preferably, between 500 mg and 3 g of biomimetic solids are administered per day. Biomimetic solids can be delivered inside liposomes or biodegradable polymers for enhanced delivery. Numerous modifications of the delivery of biomimetic solids will be obvious to those skilled in the art and are, therefore, included in this patent.

The invention is not limited by the embodiments described above which are presented as examples only but can be modified in various ways within the scope of protection defined by the appended patent claims. All references cited herein are incorporated by reference.

1-14. (canceled)

15. A method of treating a tumor in a patient comprising orally administering to the patient a pharmaceutical composition comprising a zeolite having an average particle size of about 6 microns or less.

16. The method of claim 15 wherein the zeolite is clinoptilolite.

17. The method of claim 16 wherein the clinoptilolite has a mean particle size of 250 nm.

18. The method of claim 15 wherein the pharmaceutical composition further comprises at least one of a pro-oxidant metal complex, zinc, silver, a cytokine, a cell, and a tumor antigen.

19. The method of claim 18 wherein the cell is a live vaccine cell.

20. The method of claim 18 wherein the cytokine is IL-12, GM-CSF or interferon gamma.

21. The method of claim 15 wherein the tumor is selected from the group consisting of lung cancer and colorectal cancer.

22. A method for treating a tumor in a patient comprising intravenously administering to the patient a pharmaceutical composition comprising a zeolite having a mean particle size of 250 nm.
